(19) United States
(12) Patent Application Publication
(10) Pub. No.: US 2004/0186274 A1
(43) Pub. Date: Sep. 23, 2004

(54) Methylation of histone h4 at arginine 3

(76) Inventors: C. David Allis, New York, NY (US); Scott D. Briggs, West Lafayette, IN (US); Brian D. Strahl, Chapel Hill, NC (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/482,725

(22) PCT Filed: Jul. 2, 2002
(86) PCT No.: PCT/US02/20906

Related U.S. Application Data
(60) Provisional application No. 60/302,811, filed on Jul. 3, 2001.

(51) Int. Cl. A61K 39/395; C07K 7/08; C07K 7/06

(52) U.S. Cl. 530/326; 530/327; 530/328; 530/329; 530/330

(57) ABSTRACT

The present invention relates to the generation of antibodies that bind to specific modifications of the amino terminus of histone H3 and H2A peptides. More particularly, the present invention is directed to the generation of a set of antibodies that recognize various post-tanslational modifications of a histone modification cassette SGRGK (SEQ ID NO: 1), wherein the modifications are selected from the group consisting of a phosphorylated serine, methylated arginine and acetylated lysine. Compositions comprising these antibodies are used as diagnosed and screening tools.

This application claims priority under 35 U.S.C. §119(e) to U.S. Provisional Patent Application No. 60/302,811, filed on Jul. 3, 2001, the disclosure of which is incorporated herein by reference in its entirety.

### US GOVERNMENT RIGHTS

This invention was made with United States Government support under Grant Nos. GM53512, GM20039, GM37537-14 and DK55274, awarded by the National Institutes of Health. The United States Government has certain rights in the invention.

### FIELD OF THE INVENTION

The present invention is directed to antibodies that bind to histone epitopes created by post-translational modification of the histone protein, compositions comprising such antibodies, and the use of such compositions as diagnostic and screening tools.

### BACKGROUND OF THE INVENTION

In eukaryotes, DNA is complexed with histone proteins to form nucleosomes, the repeating subunits of chromatin. This packaging of DNA imposes a severe restriction to proteins seeking access to DNA for DNA-templated processes such as transcription or replication. It is becoming increasingly clear that post-translational modifications of histone amino-termini play an important role in determining the chromatin structure of the eukaryotic cell genome as well as regulating the expression of cellular genes.

Posttranslational modifications of histone amino-termini have long been thought to play a central role in the control of chromatin structure and function. A large number of covalent modifications of histones have been documented, including acetylation, phosphorylation, methylation, ubiquitination, and ADP ribosylation, that take place on the amino terminus “tail” domains of histones. Such diversity in the types of modifications and the remarkable specificity for residues undergoing these modifications suggest a complex hierarchy of order and combinatorial function that remains unclear. Of the covalent modifications known to take place on histone amino-termini, acetylation is perhaps the best studied and appreciated. Recent studies have identified previously characterized coactivators and corepressors that acetylate or deacetylate, respectively, specific lysine residues in histones in response to their recruitment to target promoters in chromatin (See Berger (1999) Curr. Opin. Genet. Dev. 11, 336-341). These studies provide compelling evidence that chromatin remodeling plays a fundamental role in the regulation of transcription from nucleosomal templates.

Chromosomes in higher eukaryotes have historically been considered to consist of regions of euchromatin and heterochromatin, which are distinguished by the degree of condensation and level of transcriptional activity of the underlying DNA sequences. Certain regions of constitutive heterochromatin are found at or near specialized structures such as centromeres, and are comprised mostly of genetically inert repetitive sequences. In contrast, other regions that have the same primary DNA sequences can exhibit characteristics of either type of chromatin, suggesting that epigenetic factors, such as packaging of DNA by histones and chromatin associated proteins, dictate the heterochromatin status at these loci.

Although the regulation of gene expression is probably one of the more intensely studied areas of current molecular biology, fundamental to all proliferating cells, whether they divide by mitosis or meiosis, is the faithful segregation of condensed chromosomes. In 1997, it was reported that histone H3 is uniquely phosphorylated at serine 10 during mitosis and meiosis in a wide range of organisms (Hendzel et al., 1997; Wei and Allis, 1998; Wei et al., 1998). Consistent with the hypothesis that H3 phosphorylation at serine 10 (Ser10) plays an important role with mitotic chromosome condensation in vivo, mutation of the H3 gene in Tetrahymena (S10A) displays abnormal patterns of chromosome segregation leading to extensive chromosome loss during mitosis and meiosis (Wei et al., 1999). Therefore post-translational modification of histones also appear to be involved in mitotic and meiotic processes.

Through the use of antibodies that specifically recognize histones that bear post-translational modifications, applicants have been elucidating a “histone code.” In particular, evidence is emerging that histone proteins, and their associated covalent modifications, contribute to a mechanism that can alter chromatin structure, thereby leading to inherited differences in transcriptional “on-off” states or to the stable propagation of chromosomes by defining a specialized higher-order structure.

In addition to the previously described histone code, a new theme is emerging in the field that histone modifications most likely do not function in isolation. For example, phosphorylation of H3 at Ser10 appears to have a ‘split personality,’ exhibiting clear links to mitotic chromatin as well as transcriptionally-active chromatin. These results seem counter-intuitive, but they have provided an impetus to explore if other neighboring modifications act together with Ser10 Phos H3 to distinguish these responses. Recent data, collected in yeast and in mammalian cells, document that Phos (Ser10) H3 can function in some circumstances with adjacent or nearby acetylation sites (Lys9 and/or Lys14). The exact order of addition or degree of non-randomness of these events remains controversial. Nevertheless, the Phos/Acetyl di-modification of H3 seems to be part of a highly conserved gene-inductive response.

The fact that post-translational modifications of adjacent amino acid residues may interact to produce an effect has lead to the notion that certain “histone modification cassettes” exist that regulate basic genomic functions such as transcription and replication. A histone modification cassette comprises a primary histone amino acid sequence that contains two or more sites that are naturally modified under certain circumstances, wherein the post-translational modifications interact to give an specific response. One aspect of the present invention is directed to a “histone modification cassette” that is centered around a methylation site at Arg3 of histone 4.

Histone methylation is one of the least-understood post-translational modifications affecting histones. Early work suggests that H3 and H4 are the primary histones modified by methylation, and sequencing studies, using bulk histones, have shown that several lysines (e.g., 9 and 27 of H3 and 20 of H4) are often preferred sites of methylation, although species-specific differences appear to exist. Interestingly, each modified lysine has the capacity to be mono-, di-, or trimethylated, adding yet another level of variation to this post-translational “mark.” One major obstacle in understanding the function of histone methylation is the lack of information about the responsible enzymes. The demonstrations that SUV39H1, the human homologue of the Drosophila heterochromatic protein Su(var)3-9, is an H3-specific methyltransferase, and that methylation of lysine 9 (Lys 9) on histone H3 serves as a binding site for the heterochromatin protein 1 (HP1) underscore the importance of histone lysine methylation in heterochromatin function. In addition to lysine residues, methylation of histones can also occur on arginine residues. The recent demonstrations that a nuclear receptor co-activator-associated protein, CARM1, is an H3-specific arginine methyltransferase suggests that histone arginine methylation may be involved in transcriptional activation.

One aspect of the present invention is directed to antibodies that recognize specific postranlational modifications patterns of histones and other DNA associated proteins, that are associated with transcriptional or mitotic activity. More particularly, the present invention is directed to antibodies that are specific for the H2A and H4 histones methylated at the amino terminal Arginine (located at amino acid position number 3).

### SUMMARY OF THE INVENTION

The present invention is directed to antibodies that bind to specific modifications of the amino terminus of histone H3 and H2A peptides. More particularly, the present invention is directed to the generation of a set of antibodies that recognize various post-translational modifications of a histone modification cassette. These antibodies recognize various modifications of the amino acid sequence SGRGK (SEQ ID NO: 1), wherein the modifications are selected from the group consisting of a phosphorylated serine, methylated arginine and acetylated lysine. Furthermore these antibodies recognize epitopes on non-histone proteins that may be linked to human biology and disease. Compositions comprising these antibodies are used as diagnostic and screening tools.

### BRIEF DESCRIPTION OF THE DRAWINGS

FIG. 1 is a graph demonstrating an ELISA analysis of a H4 Arg 3 methyl-specific rabbit antiserum (α-H4 R3Me) using unmodified or Arg 3 methylated H4 1-9 peptides.

FIG. 2A represents data from mock-(rH4) and PRMT1-methylated [rH4(mR3)] recombinant H4 histone that was subjected to p300 acetylation in the presence of ³H-Acetyl-CoA. Samples were analyzed by Coomassie, Western (using the H4 Arg3Me specific antibody) and fluorogram. Methylation of H4 by PRMT1 stimulated its subsequent acetylation by p300

FIG. 2B represents a Triton-Acetic Acid-Urea (TAU) gel analysis of the samples used in FIG. 2A. The results demonstrate that PRMT1-methylated H4 is a better substrate for p300 when compared to non-methylated H4.

### DETAILED DESCRIPTION OF THE INVENTION

Definitions

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2-S(O)2NR—) linkage, a urea (—NHC(O)NH—) linkage, a —CH2 -secondary amine linkage, or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R 2 is selected from the group consisting of C1-C4 alkoxy, and —NR3R4 where R3 and R4 are independently selected from the group consisting of hydrogen and C1-C4 alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

As used herein, the term “conservative amino acid substitution” is defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met Leu, Ile, Val, Cys

V. Large, aromatic residues:

Phe, Tyr, Trp

As used herein, the term “purified” and like terms relate to the isolation of a molecule or compound in a form that is substantially free (i.e. are at least 60% free, preferably 75% free, and most preferably 90% free) from other components with which they are naturally associated.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The surface of such supports may be solid or porous and of any convenient shape.

The term “linked” or like terms refers to the connection between two groups. The linkage may comprise a covalent, ionic, or hydrogen bond or other interaction that binds two compounds or substances to one another.

“Operably linked” refers to a juxtaposition wherein the components are configured so as to perform their usual function. For example, control sequences or promoters operably linked to a coding sequence are capable of effecting the expression of the coding sequence.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents and includes agents approved by a regulatory agency of the US Federal government or listed in the US Pharmacopeia for use in animals, including humans. The term “carrier” refers to a diluent, adjuvant, excipient or vehicle with which an active agent is administered.

As used herein, the term “histone modification cassette” is intended to include any grouping of two or more histone modifications within a contiguous amino acid sequence of a histone tail that in combination are associated with a specific biological response. Examples of specific biological responses include but are not limited to transcriptional activation and the initiation of mitosis or meiosis.

As used herein, the term “post-translational modification cassette” is intended to include any grouping of two or more post-translational modifications of a contiguous amino acid sequence that in combination are associated with a specific biological response.

As used herein, the term “antibody” refers to a polyclonal or monoclonal antibody or a binding fragment thereof such as Fab, F(ab′)₂ and Fv fragments.

As used herein, the term “biologically active fragments” of the antibodies described herein encompasses natural or synthetic portions of the respective full-length antibody that retain the capability of specific binding to the target epitope.

As used herein, the term “parenteral” includes administration subcutaneously, intravenously or intramuscularly.

As used herein the designation Ser(P) when used in the context of an amino acid sequence, will represent a serine amino acid that has been phosphorylated.

As used herein the designation Lys(A), when used in the context of an amino acid sequence, will represent a lysine amino acid that has been acetylated.

As used herein the designation Arg(M), when used in the context of an amino acid sequence, will represent an arginine amino acid that has been methylated.

The Invention

The present invention is directed to compositions and methods for identifying transcriptionally active and inactive regions of chromatin and the use of such information for diagnostic and therapeutic purposes. The compositions comprise antibodies that are specific for certain post-translational modifications of histone proteins that have been associated with a biological state. Recent results have suggested that histones are a potential physiological targets for arginine methylation. Analysis of histones isolated from human cells revealed that some but not all H4 molecules are methylated at Arg 3. Antibodies specific to this methylation site in histone H4 have been generated and have revealed that this modification site plays a role in transcriptional activation.

To identify enzymes involved in core histone methylation, nuclear proteins from HeLa cells were separated into a nuclear extract and nuclear pellet followed by further fractionation on DEAE52 and phosphate cellulose P11 columns. The resulting fractions were assayed for methyltransferase activity using core histone octamers as substrates. By following histone methyltransferase (HMT) activity an H4-specific HMT from the nuclear pellet fraction was purified to homogeneity. Silver staining of a SDS-PAGE containing the column fractions revealed again that a 42 kDa polypeptide co-eluted with the enzymatic activity. Mass spectrometry analysis identified the 42 kDa polypeptide as the human protein arginine N-methyltransferase 1, PRMT1.

The identification of PRMT1 as one of the most abundant H4-specific HMT is surprising since only Lys 20 of H4 has been reported to be methylated in vivo, and PRMT1 is not known to be able to methylate lysine residues. To determine whether PRMT1 methylates H4 on Lys 20, core histone octamers were methylated with recombinant or native PRMT1 in the presence of S-adenosyl-L-[methyl-³H]methionine (³H-SAM). After separation by SDS-PAGE, methylated H4 was recovered and microsequenced by automated Edman chemical sequencing. Sequentially released amino acid derivatives were collected and counted by liquid scintillation revealing that Arg 3, instead of Lys 20, was the major methylation site.

To determine whether Arg3 methylation occurs in vivo, antibodies against an Arg 3-methylated histone H4 N-terminal peptide were generated. In particular, a synthetic peptide coding for the human H4 N-terminal 9 amino acids (SGRGKGGKGC*, SEQ ID NO: 15), in which the first serine was N-acetylated and residue 3 was asymmetric NG,NG-dimethylated (Bachem), was conjugated to keyhole limpet hemocyanin via a C-terminal artificial cysteine (C*) prior to rabbit immunization.

The resulting antibody reacts strongly with PRMT1 -methylated H4, and does not recognize equal amounts of recombinant H4 expressed in E. coli, indicating that the antibody is methyl-Arg 3-specific. Significantly, this same antibody also recognized histone H4 purified from HeLa cells indicating certain level of Arg 3-methylation occurs in vivo. H2A can also be weakly methylated by PRMT1 in vitro and methylated H2A can be recognized by the methy-Arg 3 antibody. The methylation site on H2A is likely to be Arg 3 because H2A has the same extreme N-terminal sequence SGRGK (SEQ ID NO: 1) as that of H4. However, the endogenous H2A methylation level has not been detected under the similar conditions.

The extreme N-terminal sequence SGRGK (SEQ ID NO: 1) of H4 and H2A is also subject to several other post-translational modifications including phosphorylation of serine 1 and acetylation of lysine 5. These three modifications interact and constitute what will be referred to as a “post-translational modification cassette.” More particularly, certain modifications of the primary sequence SGRGK (SEQ ID NO: 1) have been associated with specific biological responses, including transcriptional activation and the initiation of mitosis or meiosis. Applicants have found that antibodies generated against the modified sequence Ser(P) Gly Arg Gly Lys (SEQ ID NO: 3) serve as a mitotic marker, similar to antibodies raised against Phos Ser10 in H3. Furthermore, antibodies generated against the modified sequence Ser Gly Arg(M) Gly Lys, (SEQ ID NO: 2) and Ser Gly Arg(M) Gly Lys(A) (SEQ ID NO: 6) served as markers of transcriptional activity. Accordingly, this region of H4 appears to be involved in both transcriptional up-regulation as well as mitosis depending on the particular postranlational modification of the primary amino acid sequence.

One aspect of the present invention is directed to antigenic peptides comprising an amino acid sequence of 5 to 20, and more preferably 5-9 amino acid residues wherein the amino acid sequence is selected from the group consisting of:

| | |
|---|---|
| Ser Gly Arg(M) Gly Lys, | (SEQ ID NO: 2) |
| Ser(P) Gly Arg Gly Lys, | (SEQ ID NO: 3) |
| Ser(P) Gly Arg(M) Gly Lys, | (SEQ ID NO: 4) |
| Ser Gly Arg Gly Lys(A), | (SEQ ID NO: 5) |
| Ser Gly Arg(M) Gly Lys(A), | (SEQ ID NO: 6) |
| and | |
| Ser(P) Gly Arg(M) Gly Lys(A), | (SEQ ID NO: 7) |

wherein “Ser(P), “Arg(M)” and “Lys(A)” represents a phosphorylated serine, a methylated Arg residue and an acetylated lysine, respectively. Preferably the peptide is a purified antigenic fragment of a histone protein, or a corresponding a synthetic equivalent thereof, comprising an amino acid sequence selected from the group consisting of:

| | |
|---|---|
| Ser Gly Arg(M) Gly Lys Gly Gly Lys Gly, | (SEQ ID NO: 8) |
| Ser(P) Gly Arg Gly Lys Gly Gly Lys Gly, | (SEQ ID NO: 9) |
| Ser(P) Gly Arg(M) Gly Lys Gly Gly Lys Gly, | (SEQ ID NO: 10) |
| Ser Gly Arg Gly Lys(A) Gly Gly Lys Gly, | (SEQ ID NO: 11) |
| Ser Gly Arg(M) Gly Lys(A) Gly Gly Lys Gly, | (SEQ ID NO: 12) |
| Ser(P) Gly Arg(M) Gly Lys(A) Gly Gly Lys Gly, | (SEQ ID NO: 13) |
| and | |
| Ser Gly Arg Gly Lys Gly Gly Lys Gly. | (SEQ ID NO: 14) |

More particularly the antigenic peptide comprises a 9 to 20 amino acid sequence, and more preferably a 9 amino acid sequence, wherein the amino acid sequence comprises the sequence of SEQ ID NOs: 8-14, or an amino acid sequence that differs from SEQ ID NOs: 8-14 by a single conservative amino acid substitution at positions 6-9 (i.e. a substitution within the sequence Gly Gly Lys Gly of those peptides). In an alternative embodiment, the purified antigen comprises and amino acid sequence selected from the group of SEQ ID NOs: 1-13 linked to a suitable carrier, such as bovine serum albumin or Keyhole limpet hemocyanin.

In one preferred embodiment the antigen consists of a peptide having at least nine consecutive residues and comprising an amino acid sequence selected from the group consisting of Ser(P) Gly Arg(M) Gly Lys (SEQ ID NO: 4), Ser Gly Arg(M) Gly Lys(A) (SEQ ID NO: 6) and Ser Gly Arg(M) Gly Lys (SEQ ID NO: 2). In another embodiment the antigenic composition consists an amino acid sequence selected from the group consisting of SEQ ID NOs: 8, SEQ ID NO: 10, SEQ ID NO: 12 and derivatives of these amino acid sequences wherein the amino acid sequence contains one or more conservative amino acid substitutions at positions 6-9 (i.e. a substitution within the sequence Gly Gly Lys Gly of those peptides), and optionally a carrier protein linked to the peptide. In another embodiment, the purified antigen consists of a peptide having at least nine consecutive residues of the amino acid sequence: Ser Gly Arg Gly Lys Gly Gly Lys Gly (SEQ ID NO: 14), and derivatives of this amino acid sequence wherein the amino acid sequence contains one or more conservative amino acid substitutions, and optionally a carrier protein linked to the peptide.

The present invention also encompasses antibodies generated against the modified peptides. One method used to generate these antibodies involves administration of the respective antigens to a laboratory animal, typically a rabbit, to trigger production of antibodies specific for the antigen. Accordingly the present invention also encompasses antigenic compositions comprising a peptide comprising an amino acid sequence selected form the group consisting of the peptides of SEQ ID NO: 2 - SEQ ID NO: 14 and a pharmaceutically acceptable carrier. The composition may further comprise diluents, excipients, solubilizing agents, stabilizers and adjuvants. Carriers and diluents suitable for use with the present invention include sterile liquids such as water and oils. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions. Suitable adjuvants include alum or complete Freund's adjuvant (such as Montanide ISA-51).

The dose and regiment of antigen administration necessary to trigger antibody production as well as the methods for purification of the antibody are well known to those skilled in the art. Typically, such antibodies can be raised by administering the antigen of interest subcutaneously to New Zealand white rabbits which have first been bled to obtain pre-immune serum. The antigens can be injected at a total volume of 100 ul per site at six different sites. Each injected material will contain synthetic surfactant adjuvant pluronic polyols, or pulverized acrylamide gel containing the protein or polypeptide after SDS-polyacrylamide gel electrophoresis.

The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., editors, Antibodies: A Laboratory Manual (1988), which is hereby incorporated by reference. The specificity of antibodies may be determined by enzyme-linked immunosorbent assay or immunoblotting, or similar methods known to those skilled in the art.

On aspect of the present invention is directed to antibodies that specifically bind to a polypeptide comprising an amino acid sequence selected from the group consisting of:

Ser Gly Arg Gly Lys, (SEQ ID NO: 1)

Ser Gly Arg(M) Gly Lys, (SEQ ID NO: 2),

Ser(P) Gly Arg Gly Lys, (SEQ ID NO: 3),

Ser(P) Gly Arg(M) Gly Lys, (SEQ ID NO: 4),

Ser Gly Arg Gly Lys(A), (SEQ ID NO: 5),

Ser Gly Arg(M) Gly Lys(A), (SEQ ID NO: 6),

and Ser(P) Gly Arg(M) Gly Lys(A), (SEQ ID NO: 7). In one embodiment the antibodies of the present invention specifically bind to a polypeptide comprising an amino acid sequence selected from the group consisting of

| | |
|---|---|
| Ser Gly Arg(M) Gly Lys Gly Gly Lys Gly, | (SEQ ID NO: 8) |
| Ser(P) Gly Arg Gly Lys Gly Gly Lys Gly, | (SEQ ID NO: 9) |
| Ser(P) Gly Arg(M) Gly Lys Gly Gly Lys Gly, | (SEQ ID NO: 10) |
| Ser Gly Arg Gly Lys(A) Gly Gly Lys Gly, | (SEQ ID NO: 11) |
| Ser Gly Arg(M) Gly Lys(A) Gly Gly Lys Gly, | (SEQ ID NO: 12) |
| Ser(P) Gly Arg(M) Gly Lys(A) Gly Gly Lys Gly, | (SEQ ID NO: 13) |
| and | |
| Ser Gly Arg Gly Lys Gly Gly Lys Gly. | (SEQ ID NO: 14) |

More particularly, the antibody specifically binds to a polypeptide selected from the group consisting of

| | |
|---|---|
| Ser Gly Arg(M) Gly Lys Gly Gly Lys Gly, | (SEQ ID NO: 8) |
| Ser(P) Gly Arg(M) Gly Lys Gly Gly Lys Gly, | (SEQ ID NO: 10) |
| Ser Gly Arg(M) Gly Lys(A) Gly Gly Lys Gly, | (SEQ ID NO: 12) |
| and | |
| Ser(P) Gly Arg(M) Gly Lys(A) Gly Gly Lys Gly, | (SEQ ID NO: 13) |

In one embodiment the present invention is directed to an antibody that specifically binds to the polypeptide Ser Gly Arg(M) Gly Lys Gly Gly Lys Gly (SEQ ID NO: 8), or an antibody that specifically binds to the polypeptide Ser(P) Gly Arg(M) Gly Lys Gly Gly Lys Gly (SEQ ID NO: 10) or an antibody that specifically binds to the polypeptide Ser Gly Arg(M) Gly Lys(A) Gly Gly Lys Gly (SEQ ID NO: 12) or an antibody that specifically binds to the polypeptide Ser Gly Arg Gly Lys Gly Gly Lys Gly (SEQ ID NO: 14). As used herein, an antibody that binds specifically to a target antigen is an antibody that will produce a detectable signal in the presence of the target antigen but will not cross react with other non-target antigens (i.e. produces no detectable signal) under the identical conditions used to detect the target antigen. For example the monoclonal antibody generated against Ser Gly Arg(M) Gly Lys Gly Gly Lys Gly (SEQ ID NO: 8) will not bind to the sequence Ser Gly Arg Gly Lys Gly Gly Lys Gly (SEQ ID NO: 14) or any of the other peptides of SEQ ID NO: 9-13, when optimal conditions are used.

In one preferred embodiments the antibody for the target modified peptide is a monoclonal antibody. Monoclonal antibody production may be effected using techniques well-known to those skilled in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. One embodiment of the invention is directed to a hybridoma cell line which produces monoclonal antibodies which bind one of the target antigens of SEQ ID NO: 1-SEQ ID NO: 14. A description of the theoretical basis and practical methodology of fusing such cells is set forth in Kohler and Milstein, Nature, 256:495 (1975), which is hereby incorporated by reference.

In addition to whole antibodies, fragments of antibodies can retain binding specificity for a particular antigen. Antibody fragments can be generated by several methods, including, but not limited to proteolysis or synthesis using recombinant DNA technology. An example of such an embodiment is selective proteolysis of an antibody by papain to generate Fab fragments, or by pepsin to generate a F(ab′)₂ fragment. These antibody fragments can be made by conventional procedures, as described in J. Goding, Monoclonal Antibodies: Principles and Practice, pp. 98-118 (N.Y. Academic Press 1983), which is hereby incorporated by reference. Other fragments of the present antibodies which retain the specific binding of the whole antibody can be generated by other means known to those skilled in the art.

The antibodies or antibody fragments of the present invention can be combined with a carrier or diluent to form a composition. These compositions can be used in standard Molecular Biology techniques such as Western blot analyses, immunofluorescence, and immunoprecipitation. In accordance with one embodiment the antibodies of the present invention are labeled for use in diagnostics or therapeutics. It is not intended that the present invention be limited to any particular detection system or label. The antibody may be labeled with a radioisotope, such as ³⁵S, ¹³¹I, ¹¹¹In, ¹²³I, ^{99m}Tc, ³²P, ¹²⁵I, ³H, ¹⁴C, and ¹⁸⁸Rh, or a non-labeling reagent including fluorescent labels, such as fluorescein and rhodamine, or other non-isotopic labeling reagents such as biotin or digoxigenin. Antibodies containing biotin may be detected using “detection reagents” such as avidin conjugated to any desirable label such as a fluorochrome. Additional labels suitable for use in accordance with the present invention include nuclear magnetic resonance active labels, electron dense or radiopaque materials, positron emitting isotopes detectable by a positron emission tomography (“PET”) scanner, chemilluminescers such as luciferin, and enzymatic markers such as peroxidase or phosphatase. In one embodiment the histone specific antibodies of the present invention are detected through the use of a secondary antibody, wherein the secondary antibody is labeled and is specific for the primary antibody. Alternatively, the antibodies of the present invention may be directly labeled with a radioisotope or fluorochrome such as FITC or rhodamine; in such cases secondary detection reagents may not be required for the detection of the labeled probe. In accordance with one preferred embodiment the antibody is labeled with a fluorophore or chromophore using standard moieties known in the art.

In accordance with one embodiment of the present invention a method of detecting modified histone proteins is provided, and more specifically modifications of H4 or H2A histones at one of the first 5 amino acids of the amino terminus of the histone. The method comprises the steps of contacting histone proteins with an antibody, wherein the antibody specifically binds only to the H4 or H2A histones that comprise a modified sequence selected from the group consisting of:

| | |
|---|---|
| Ser Gly Arg(M) Gly Lys, | (SEQ ID NO: 2) |
| Ser(P) Gly Arg Gly Lys, | (SEQ ID NO: 3) |
| Ser(P) Gly Arg(M) Gly Lys, | (SEQ ID NO: 4) |
| Ser Gly Arg Gly Lys(A), | (SEQ ID NO: 5) |
| Ser Gly Arg(M) Gly Lys(A), | (SEQ ID NO: 6) |
| and | |
| Ser(P) Gly Arg(M) Gly Lys(A). | (SEQ ID NO: 7) |

More preferably the antibody specifically binds to a H4 or H2A histone that comprises the amino acid sequence Ser Gly Arg(M) Gly Lys (SEQ ID NO: 2), or Ser(P) Gly Arg(M) Gly Lys (SEQ ID NO: 4), or Ser Gly Arg(M) Gly Lys(A) (SEQ ID NO: 6).

The antibodies of the present invention can be linked to a detectable label using standard reagents and techniques known to those skilled in the art. For example, see Wensel and Meares, Radioimmunoimaging and Radioimmunotherapy, Elsevier, N.Y. (1983), which is hereby incorporated by reference, for techniques relating to the radiolabeling of antibodies. See also, D. Colcher et al., “Use of Monoclonal Antibodies as Radiopharmaceuticals for the Localization of Human Carcinoma Xenografts in Athymic Mice,” Meth. EnzvmQL, 121: 802-816 (1986), which is hereby incorporated by reference.

Applicants have discovered that postranlational modifications of the first five amino acids of the amino terminus of histone H4, particularly at serine 1, arginine 3 and lysine 5, correlates with the activation and inactivation gene expression for genes in proximity to the modified histones. In particular, histone H4 that contains a methylated Arg3 has been associated with transcriptional activity, particularly when the methylation of Arg3 is accompanied by the acetylation of Lys5. In addition, chromatin containing histone H4, wherein the H4 contains a methylated Arg3 and a phosphorylated Ser1, is associated with transcriptionally silent regions and serves as a marker for mitotic activity. Accordingly, in one embodiment of the present invention, antibodies specific for the methylated Arg3/acetylated Lys5 histone H4 can be used to detect transcriptionally active regions of chromatin, and antibodies specific for the methylated Arg3/phosphorylated Ser1 histone H4 can be used to detect inactive regions of chromatin and mitotically active cells.

In accordance with one embodiment, a method of detecting transcriptionally active regions of chromatin is provided. The method comprises the steps of contacting a chromatin containing sample with an antibody that specifically binds to the sequence Ser Gly Arg(M) Gly Lys(A) Gly Gly Lys Gly (SEQ ID NO: 12) under conditions suitable for specific binding and for a time sufficient to allow the antibody to bind to its target. In one preferred embodiment either the antibody or the chromatin is bound to a solid support. The sample is then washed with a buffered solution to remove unbound and non-specific bond antibody from the sample, and chromatin regions that retain the antibody are identified as transcriptionally active regions of chromatin. In one embodiment the antibody is directly labeled with a detectable label (such as a radioisotope or fluorophore) or in an alternative embodiment a secondary antibody is used to detect the antibody, wherein the secondary antibody is a labeled anti-immunoglobulin antibody specific for the primary antibody.

In another embodiment of the present invention a method of detecting mitotically active cells is provided. The method comprises the steps of obtaining a sample of cells (including a biopsy sample) and preparing the sample cells for histological analysis using standard techniques. The population of cells is then contacted with an antibody that specifically binds to the sequence Ser(P) Gly Arg(M) Gly Lys Gly Gly Lys Gly (SEQ ID NO: 10) under conditions suitable for specific binding and for a time sufficient to allow the antibody to bind to its target. In one preferred embodiment either the antibody or the cells are bound to a solid support. The sample is then washed with a buffered solution to remove unbound and non-specific bond antibody from the sample, and the mitotically active cells are identified as those cells with antibody bound to them. By comparing the number of actively dividing cells in a sample a disease characterized by inappropriate cell growth (either excessive or insufficient cell growth) can be identified. Thus the method can be used as a diagnostic for identifying disease states. In one embodiment the antibody is directly labeled with a detectable label (such as a radioisotope or fluorophore) or in an alternative embodiment a secondary antibody is used to detect the antibody, wherein the secondary antibody is a labeled anti-immunoglobulin antibody specific for the primary antibody.

The use of the modification SGRGK cassette antibodies (i.e. SEQ ID NOs: 2-13) in chromatin immunoprecipitation (chromatin IP) assays (and more particularly antibodies against the peptides Ser(P) Gly Arg(M) Gly Lys Gly Gly Lys Gly (SEQ ID NO: 10) and Ser Gly Arg(M) Gly Lys(A) Gly Gly Lys Gly (SEQ ID NO: 12) is one way to enrich for genomic DNA corresponding to the epigenetic ‘ON/OFF’ state of the human genome (and other genomes as well). By combining chromatin immunoprecipitated DNA with current genomic microarray technology (on chips), one has the potential to survey any portion of the human (or other) genome as to their on/off state through the ‘histone code’. More particularly, the use of antibodies directed to amino acid sequences Ser(P) Gly Arg(M) Gly Lys Gly Gly Lys Gly Leu, (SEQ ID NO: 10) and Ser Gly Arg(M) Gly Lys(A) Gly Gly Lys Gly Leu, (SEQ ID NO: 12), will allow for the detection of chromatin associated with transcriptionally inactive and transcriptionally on, respectively. After segregating the chromatin into heterochromatin and euchromatin, DNA chips can be used to assess what genes are being expressed/suppressed in a particular tissue.

In one embodiment, immunoprecipitation of chromatin will be used to map the location of active genes at a genome-wide level through the use of microarrays. For example, in one preferred embodiment the method of comparing the two pools of immunoprecipitated chromatin (i.e. the immunoprecipitated chromatin from diseased vs healthy tissues) comprises the use of a gene chip, DNA microarray, or a proteomics chip using standard techniques known to those skilled in the art. For example any of the systems described in WO 01/16860,WO 01/16860, WO 01/05935, WO 00/79326, WO 00/73504, WO 00/71746 and WO 00/53811 (the disclosures of which are expressly incorporated herein) are suitable for use in the present invention. Preferably the chip will contain an ordered array of known compounds (such as known DNA sequences, antibodies or other ligands) so that interaction of the immunoprecipitated chromatin (or the individual DNA or protein components recovered from the immunoprecipitated chromatin) at a specific location of the chip will identify, and allow for the isolation of, DNA sequences or proteins associated with the immunoprecipitated chromatin.

In accordance with one embodiment a method is provided for detecting chromatin alterations that are associated with a disease state. The term “disease state” is intended to encompass any condition that is associated with an impairment of the normal state of a living animal or plant including congenital defects, pathological conditions such as cancer, and responses to environmental factors and infectious agents (bacterial, viral, etc.). The method comprises the steps of isolating chromatin from both normal and diseased tissue, contacting the two pools of chromatin with an antibody that specifically binds to a polypeptide comprising an amino acid sequence selected from the group consisting of

Ser Gly Arg(M) Gly Lys Gly Gly Lys Gly (SEQ ID NO: 8),

Ser(P) Gly Arg Gly Lys Gly Gly Lys Gly (SEQ ID NO: 9),

Ser(P) Gly Arg(M) Gly Lys Gly Gly Lys Gly (SEQ ID NO: 10),

Ser Gly Arg Gly Lys(A) Gly Gly Lys Gly (SEQ ID NO: 11),

Ser Gly Arg(M) Gly Lys(A) Gly Gly Lys Gly (SEQ ID NO: 12),

Ser(P) Gly Arg(M) Gly Lys(A) Gly Gly Lys Gly (SEQ ID NO: 13),

and Ser Gly Arg Gly Lys Gly Gly Lys Gly (SEQ ID NO: 14), and more preferably, an antibody that specifically binds to a polypeptide comprising an amino acid sequence selected from the group consisting of

Ser Gly Arg(M) Gly Lys Gly Gly Lys Gly (SEQ ID NO: 8),

Ser(P) Gly Arg(M) Gly Lys Gly Gly Lys Gly (SEQ ID NO: 10), and

Ser Gly Arg(M) Gly Lys(A) Gly Gly Lys Gly (SEQ ID NO: 12); and

comparing the staining pattern of the chromatin isolated from normal tissue to that of the diseased tissue. More particularly, the staining pattern of the two pools of chromatin isolated by immunoprecipitation can be compared through the use of any standard technique, including Coomassie stained gels, Western blot analysis, Southern blot analysis, or sequencing of the protein or nucleic acid sequences associated with the respective immunoprecipitated chromatin. Of particular interest are those sequences that are present in only one of the two pools of chromatin (or present in substantially lower amounts in one pool vs the other).

In one embodiment chromatin immunoprecipitation using the antibodies of the present invention, in combination with differential screening techniques, is used to isolate unique tumor suppressor genes or other markers of a disease state. In this method chromatin is immunoprecipitated from two separate pools of chromatin, isolated from normal tissue and diseased tissue, respectively. The antibody used would preferably be an antibody that specifically binds to a polypeptide comprising an amino acid sequence selected from the group consisting of

Ser Gly Arg(M) Gly Lys Gly Gly Lys Gly (SEQ ID NO: 8),

Ser(P) Gly Arg(M) Gly Lys Gly Gly Lys Gly (SEQ ID NO: 10), and

Ser Gly Arg(M) Gly Lys(A) Gly Gly Lys Gly (SEQ ID NO: 12). The chromatin associated proteins and nucleic acids are then recovered from the respective immunoprecipitated pools of chromatin and compared using standard techniques. In one embodiment the two pools of recovered protein/nucleic acids are compared using standard techniques. Proteins present in one pool of recovered proteins but absent (or substantially reduced in quantity) or otherwise altered in the other pool of proteins will be identified, again using standard techniques, such as microsequencing or Tandem Mass Spectroscopic Analysis. Similarly, nucleic acid sequences will be analyzed by standard techniques including PCR, gel electrophoresis, nucleic acid sequencing, nucleic acid hybridization analysis and Tandem Mass Spectroscopic Analysis to identify nucleic acid sequences present in one pool of recovered sequences but absent (or substantially reduced in quantity) the other pool of recovered nucleic acid sequences.

Using the antibodies of the present invention alterations in chromatin structure that are associated with a given disease state can be detected. For example, the antibodies can be used as a diagnostic to detect alterations of chromatin structure that are associated with alterations in expression patterns (i.e. differences in heterochromatin vs euchromatin patterns relative to predominant native patterns). Alterations in chromatin structure for a specific region of chromatin may be diagnostic of a particular disease state. For example, conversion of a normally euchromatic region of the genome to heterochromatin may represent the suppression of a tumor suppressor gene that is indicative of cancer or a pre-cancer state. Similarly the conversion of a region of heterochromatin to euchromatin may be associated with the inappropriate or overexpression of a gene that has deleterious effects on the host celvorganism.

The present invention is also directed to a method of using broad-based differential screening techniques to isolate nucleic acid regions that have altered expression patterns in diseased tissues. For example, chromatin can be isolated from diseased tissues and compared to chromatin isolated from healthy tissues to determine if there are any differences in the chromatin structure (i.e. changes in heterochromatin vs. euchromatin) that are associated with the disease state. Such differences in chromatin structure may represent suppression or overexpression of genes that play a direct or indirect role in the disease. The antibodies directed against the peptide sequences Ser(P) Gly Arg(M) Gly Lys Gly Gly Lys Gly (SEQ ID NO: 10), and Ser Gly Arg(M) Gly Lys(A) Gly Gly Lys Gly (SEQ ID NO: 12) can be used to detect such changes in chromatin structure and help identify genes that are associated with the disease state. The identification of such genes will assist in designing more effective therapies for treating the disease.

In one embodiment the method for detecting alterations in chromatin structure associated with a particular disease comprises chromatin immunoprecipitation assays, using modification-specific histone antibodies. This process allows for the analysis of a wide range of DNA-templated processes that are governed by the chromatin environment. More particularly, the method comprises the steps of isolating chromatin from both diseased tissue and healthy tissue, fragmenting the DNA (preferably by sonification), and immunoprecipitating chromatin using an antibody that specifically binds to an amino acid sequence selected from the group consisting of Ser(P) Gly Arg(M) Gly Lys Gly Gly Lys Gly (SEQ ID NO: 10), and Ser Gly Arg(M) Gly Lys(A) Gly Gly Lys Gly (SEQ ID NO: 12), and comparing the chromatin (and the associated DNA sequences) immunoprecipitated from the healthy tissue relative to the diseased tissue. Comparison of the two pools of immunoprecipitated chromatin will allow for the identification of differences between diseased and healthy tissues.

It has recently been reported that nucleosomes can be detected in the serum of healthy individuals. Furthermore, the serum concentrations of nucleosomes is considerably higher in patients suffering from benign and malignant diseases (Holdenrieder et al., Int J Cancer, 95(2): 114-120 (Mar 20, 2001)). Presumably, the high concentration of nucleosomes in tumor bearing patients derives from apoptosis, which occurs spontaneously in proliferating tumors. Thus, the presence of elevated levels of nucleosomes in the blood of patients can serve as a diagnostic of diseases associated with enhanced cell death (Holdenrieder et al., Anticancer Res, 19(4A): 2721-2724 (1999)).

In accordance with one embodiment a serum sample can be isolated from an individual and screened with the antibodies of the present invention as diagnostic procedure to detect various disease states. First of all the antibodies can be used to determine if an abnormal concentration of nucleosomes are present in the sample. Furthermore the detection of a particular histone modifications in the blood of an individual by itself may serve as a diagnostic for a particular disease state. In addition further analysis can be conducted by immunoprecipatating nucleosomes using the present antibodies and analyzing the proteins and nucleic acid sequences associated with the immunoprecipitated chromatin.

Because the antibodies of the present invention have the potential for use in humans as diagnostic and therapeutic agents, one embodiment of the present invention is directed to humanized versions of these antibodies. Humanized versions of the antibodies are needed for therapeutic applications because antibodies from non-human species may be recognized as foreign substances by the human immune system and neutralized such that they are less useful. Humanized antibodies are immunoglobulin molecules comprising a human and non-human portion. More specifically, the antigen combining region (variable region) of a humanized antibody is derived from a non-human source (e.g. murine) and the constant region of the humanized antibody is derived from a human source. The humanized antibody should have the antigen binding specificity of the non-human antibody molecule and the effector function conferred by the human antibody molecule. Typically, creation of a humanized antibody involves the use of recombinant DNA techniques.

In accordance with one embodiment, the antibodies of the present invention are attached to a solid support and used to immunoprecipitate chromatin. The antibodies of the present invention can also be linked to an insoluble support to provide a means of isolating euchromatin or heterochromatin from cells. The support may be in particulate or solid form and could include, but is not limited to: a plate, a test tube, beads, a ball, a filter or a membrane. Methods for fixing antibodies to insoluble supports are known to those skilled in the art. In one embodiment an antibody of the current invention is fixed to an insoluble support that is suitable for use in affinity chromatography. Immunoprecipitation of chromatin will be used in one embodiment of the invention to map the location of DNA-binding proteins at a genome-wide level through the use of microarrays. In addition, chromatin immunoprecipitation assays, using modification-specific histone antibodies, can be used to analyze a wide range of DNA-templated processes that are governed by the chromatin environment.

The key to this technology is the use of antibodies specific to various modification as they relate to the histone code. Applying this to human and other genomes would lay the foundation of epigenomics. For example, antibodies specific for the Ser(P) Gly Arg(M) Gly Lys cassette vs. the Ser Gly Arg(M) Gly Lys(A) cassette may be a ‘phos/acetyl’ switch that regulates differentiation vs. proliferation.

Obtaining this information may prove invaluable in determining the on/off state of key tumor suppressor or oncogenic proteins in various human cancers. The Ser Gly Arg Gly Lys cassette is present in non-histone proteins. For example, the t(8;21) translocation type acute myeloid leukemia is an acute myeloid leukemia (hereinafter referred to as “AML”) which accompanies translocation of a gene on chromosome 8 to chromosome 21. The translocated gene, AMLI, contains the sequence Ser Gly Arg Gly Lys.

Knowing how the epigenetic marking associated with this peptide sequence corresponds to genomic DNA will also guide the ability to produce transgenic animals and plants where one often finds that most transgenic DNA enters a ‘bad’ chromatin environment and is silenced. Thus, the implications for knowing how to better ‘guide’ DNA into a ‘good’ chromatin environment (i.e. chromatin associated with H4 having the Ser Gly Arg(M) Gly Lys(A) modification) for animal and plant transgenic work are high. In humans, this would impact on gene therapy issues as well (if the gene of interest isn't expressed what good is it to the patient).

In one embodiment of the present invention a kit is provided for detecting euchromatin and heterochromatin. The kit comprises an antibody that specifically binds to an amino acid sequence selected from the group consisting of:

Ser Gly Arg(M) Gly Lys, (SEQ ID NO: 2),

Ser(P) Gly Arg Gly Lys, (SEQ ID NO: 3),

Ser(P) Gly Arg(M) Gly Lys, (SEQ ID NO: 4),

Ser Gly Arg Gly Lys(A), (SEQ ID NO: 5),

Ser Gly Arg(M) Gly Lys(A), (SEQ ID NO: 6),

and Ser(P) Gly Arg(M) Gly Lys(A), (SEQ ID NO: 7). More particularly the kit comprises and antibody the specifically binds to an amino acid sequence selected from the group consisting of

Ser Gly Arg(M) Gly Lys Gly Gly Lys Gly (SEQ ID NO: 8),

Ser(P) Gly Arg(M) Gly Lys Gly Gly Lys Gly (SEQ ID NO: 10),

and Ser Gly Arg(M) Gly Lys(A) Gly Gly Lys Gly (SEQ ID NO: 12). In one embodiment the antibodies are attached to a solid support, wherein the support is either a monolithic solid or is in particular form. In one preferred embodiment the antibodies are monoclonal antibodies and in a further embodiment the antibodies are labeled. To this end, the antibodies of the present invention can be packaged in a variety of containers, e.g., vials, tubes, microtiter well plates, bottles, and the like. Other reagents can be included in separate containers and provided with the kit; e.g., positive control samples, negative control samples, buffers, cell culture media, etc.

The kits of the present invention may further comprise reagents for detecting the monoclonal antibody once it is bound to the, target antigen. Optionally, reagents (pepsin, dilute hydrochloric acid) for treating cells or tissue to render nuclear proteins accessible for immunological binding may also be included, as may immunofluorescent detection reagents (an anti-immunoglobulin antibody derivatized with fluorescein or rhodamine, or a biotinylated anti-immunoglobulin antibody together with avidin or streptavidin derivatized with fluorescein or rhodamine), immunohistochemical or immunocytochemical detection reagents (an anti-immunoglobulin antibody derivatized with alkaline phosphatase or horseradish peroxidase, or a biotinylated anti-immunoglobulin antibody together with avidin or streptavidin derivatized with alkaline phosphatase or horseradish peroxidase). In one embodiment, the kit includes one or more reagents for immunoperoxidase staining (an anti-immunoglobulin antibody derivatized with horseradish peroxidase, or a biotinylated anti-immunoglobulin antibody together with avidin or streptavidin derivatized with horseradish peroxidase), together with a chromogenic substrate therefor (e.g., diaminobenzidine).

In an alternative embodiment a kit comprising the peptide Ser Gly Arg Gly Lys Gly Gly Lys Gly Leu (SEQ ID NO: 14), optionally attached to an insoluble support, can be provided for use in an assay to determine if a sample has methylase, kinase or acetylase activity. In one embodiment, the peptide is used to detect the level of PRMT1 activity of a given sample. The method comprises contacting the peptide with the sample for a predetermined length of time and then detecting the amount of methylation has occurred on the peptide substrate through the use of an antibody that binds only to the methylated peptide.

This assay can also be used in a method of screening for potential inhibitors of methylase, kinase or acetylase activity. For example, in one embodiment a method of screening for inhibitors of arginine methyl transfer activity comprises the steps of providing a sample that comprises the methylase and a substrate that is methylated by said methylase, adding a potential inhibitor of the methylase to the sample, contacting the sample with an antibody that binds specifically to the methylated substrate, but not the non-methylated substrate. In one embodiment the antibody is specific for the peptide Ser Gly Arg(M) Gly Lys Gly Gly Lys Gly (SEQ ID NO: 8). Quantifying the amount of antibody bound to the peptide is a direct correlation of the level activity of the methylase in the sample. In one preferred embodiment the methylase activity to be detected is PRMT 1.

### EXAMPLE 1

Identification of H3 Arginine Methylation

Recent reports showing that CARM1 and PRMT1 methylate histones in vitro and contain transcriptional coactivator function suggest that histones are a potential physiological targets for arginine methylation. However, clear evidence for the existence of arginine methylation on histones has been lacking. To identify sites of arginine methylation on mammalian histones in vivo, histones isolated from asynchronously growing human 293T cells were individually purified by reverse-phase high-performance liquid chromatography (RP-HPLC), digested by chymotrypsin, and the resulting peptides examined by nano-HPLC microelectrospray ionization tandem mass spectrometry. Collision-activated dissociation (CAD) spectra of human H4 N-terminal chymotryptic peptides revealed an addition of a methyl group to the Arg 3 residue, indicating N G-monomethylation. The predicted b-and y-type ion series of the H4 peptide in which Arg 3 is monomethylated were observed. Furthermore, other CAD spectra show H4 N-terminal peptides lacking methylation at Arg 3, supporting the above characterization and indicating that some but not all H4 molecules are methylated at Arg 3.

While arginine residues are capable of being methylated at either or both of their two terminal guanidino nitrogen groups generating N G-monomethylarginine, symmetric N G, N ′G-dimethylarginine or asymmetric NG, NG-dimethylarginine, the analyses did not reveal the presence of dimethylarginine at H4 Arg 3. However, these data do not exclude the possibility that H4 Arg 3 dimethylation exists.

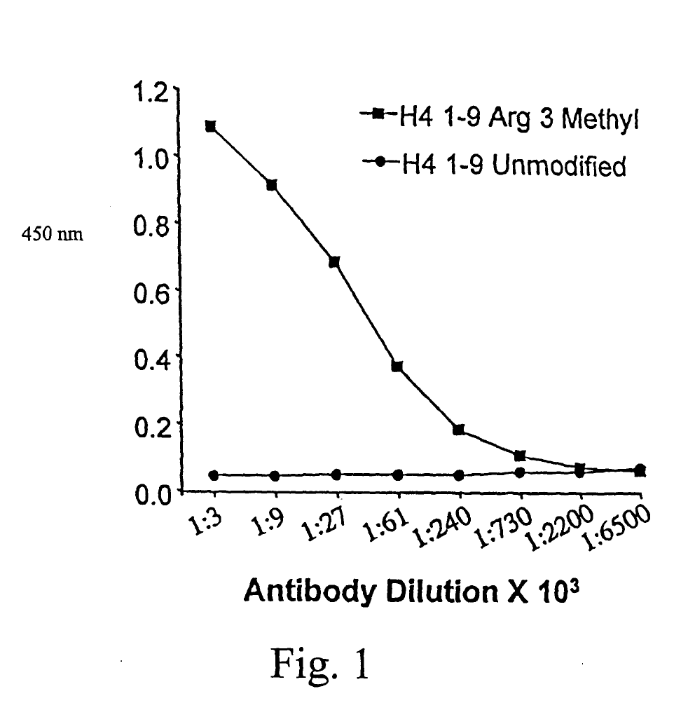

An antibody specific to this methylation site in histone H4 was generated using rabbits immunized with a H4 1-9 synthetic peptide in which Arg 3 was NG ,NG-dimethylated. Specificity of this antiserum to methylated Arg 3 was verified by ELISA using N-terminal H4 1-9 peptides that were either unmodified or methylated at Arg 3 (FIG. 1C). To examine the conservation of Arg 3 H4 methylation in vivo, histones isolated from multiple eukaryotic organisms were probed with the H4 Arg 3 methyl-specific antiserum (hereafter α-H4 R3Me). Immunoblot analyses revealed the presence of Arg 3 methylation on H4 in all histones tested with the exception of Tetrahymena which contains a divergent extreme amino-terminal H4 tail lacking Arg 3 and recombinant Xenopus H4. Together with the mass spectrometric analyses, these data suggest that the α-H4 R3Me antibody recognizes both mono-and dimethylarginine. These results clearly demonstrate the in vivo existence of arginine methylation in a broad range of eukaryotic histones. Given this conservation H4 Arg 3 methylation may play an important role in histone metabolism.

### EXAMPLE 2

Identification of the H3 Arginine Methylation Enzyme

To identify enzymes involved in core histone methylation, nuclear proteins from HeLa cells were separated into a nuclear extract and nuclear pellet followed by further fractionation on DEAE52 and phosphate cellulose P11 columns. The resulting fractions were assayed for methyltransferase activity using core histone octamers as substrates. In particular, column fractions or recombinant human protein arginine N-methyltransferase 1 (PRMT1) was incubated with core histone octamers, recombinant H4, or H4 tail peptides in a total volume of 30 ul containing 20 mM Tris-HCl (pH 8.0), 4 mM EDTA, 1 mM PMSF, 0.5 mM DTT, and 1.5 ul ³H-SAM (15 Ci/mmol; NEN Life Science Products) at 30° C. for 1 hour. Reactions were stopped by the addition of SDS loading buffer followed by electrophoresis in an 18% SDS-PAGE. After Coomassie staining and destaining, gels were treated with Entensify (NEN Life Science Products) and dried before exposing to X-ray Film.

Multiple methyltransferase activities with distinctive specificity for histones H3 and H4 were detected. By following histone methyltransferase (HMT) activity, an H4-specific HMT was purified from the nuclear pellet fraction to homogeneity. Analysis of the column fractions derived from the Hydroxyapatite column indicated that the peak of the enzymatic activity eluted in fraction 14 and trailed through fraction 26. Silver staining of a SDS-PAGE containing the column fractions revealed that a polypeptide of 42 kDa co-eluted with the enzymatic activity. To confirm this result, the same input was loaded onto a gel-filtration Superose-200 column. Analysis of the column fractions indicated that the peak of the enzymatic activity eluted around 330 kDa between fractions 38-41. Silver staining of a SDS-PAGE containing the column fractions revealed again that a 42 kDa polypeptide co-eluted with the enzymatic activity. Mass spectrometry analysis identified the 42 kDa polypeptide as the human protein arginine N-methyltransferase 1, PRMT 1.

Since the HMT activity eluted around 330 kDa and only co-eluted with PRMT1, it is likely that PRMT1 functions as a homo-oligomer. This was verified by the demonstration that recombinant PRMT1 fractionated the same as the endogenous PRMT1, as a 330 kDa complex. Therefore, we conclude that PRMT1 functions as an H4-specific HMT in the form of homo-oligomer.

The identification of PRMT1 as one of the most abundant H4-specific HMT is surprising since only Lys 20 of H4 has been reported to be methylated in vivo, and that PRMT1 is not known to be able to methylate lysine residues. Instead, PRMT1 and its yeast homologue have been reported to mainly methylate arginine of certain RNA-binding proteins.

To determine whether PRMT1 methylates H4 on Lys 20, core histone octamers were methylated with recombinant or native PRMT 1 in the presence of S-adenosyl-L-[methyl-³H]methionine (³H-SAM). After separation by SDS-PAGE, methylated H4 was recovered and microsequenced by automated Edman chemical sequencing. Sequentially released amino acid derivatives were collected and counted by liquid scintillation revealing that Arg 3, instead of Lys 20, was the major methylation site. Comparison of the ability of PRMT1 to methylate H4 tail peptides with or without a mutation on Lys 20 showed no difference, confirming that Lys 20 is not a site for PRMT1 methylation.

To determine whether PRMT1 is responsible for this site-specific Arg 3 methylation in vivo, PRMT1 was over-expressed in cells to determine if there was a corresponding increase the Arg 3 methylation level. Over-expression of PRMT1 resulted in an increase in Arg 3 methylation. To confirm this result, core histones from PRMT1^{+/+} and PRMT1^{-/-} embryonic stem (ES) cells were purified and compared for their Arg 3 methylation level. Inactivation of the Prmt1 gene results in a dramatic decrease in the Arg 3 methylation level indicating that histone H4 is likely an in vivo substrate for PRMT1.

In fact, it appears that mitotic Arg3 methylation of H4 may occur slightly before Ser1 gets phosphorylated during the synchronous progression of mitotic divisions in the slime mold, Physarum. It also has been observed that Arg3 methyl H4 is enriched on the inactive X chromosome in mouse ES cells (E. Heard, unpublished). Collectively, these data suggest that these closely neighboring modifications, defining a modification cassette, work together to mark the H4 tail towards a more condensed chromatin state in many diverse biological situations. Therefore the modified sequence Ser(P) Gly Arg(M) Gly Lys (SEQ ID NO: 4) is associated with transcriptional inactivity and is believed to have utility as a marker for mitosis.

### EXAMPLE 3

Further Analysis of the H3 Arginine Methylation Enzyme

To further determine the identity of the H4 Arg 3 methyltransferase, the substrate and site specificity of PRMT1, which was previously shown to methylate H4 in a mixture of free calf thymus histones, was examined. Purified recombinant GST-PRMT1 was incubated with core histones isolated from chicken, Tetrahymena and human 293T cells in the presence of S-Adenosyl-L-[methyl-₃H]methionine (₃H-AdoMet), and reaction products were analyzed by SDS-PAGE and fluorography. Results showed that GST-PRMT1 efficiently methylates chicken and human 293T H4 from a mixture of core histones. As expected, GST-PRMT1 was unable to methylate H4 from Tetrahymena, suggesting Arg 3 is the major, if not exclusive, site of PRMT1 methylation under these assay conditions. No methylation of histones was observed in the absence of GST-PRMT1. Under these reaction conditions, mammalian H2A was also found to be weakly methylated, a result consistent with earlier findings.

To determine definitively which arginine residue(s) in the H4 amino terminus is methylated by PRMT1,RP-HPLC purified H4 from labeling reactions using purified GST-PRMT1 and chicken core histones was microsequenced and ³H-incorporation associated with each cycle determined. Microsequence analysis shows that Arg 3 is the exclusive site of methylation in the H4 tail. In agreement, recombinant Xenopus H4 (rH4)reacted in the presence of GST-PRMT1 was strongly methylated at Arg 3 by immunoblot analysis using the α-H4 R3Me antibody. In contrast, rH4 from reactions lacking GST-PRMT1 was not immunoreactive. Nano-HPLC microelectrospray ionization mass spectrometric analyses of the identical reactions above revealed that the exclusive methylation product at Arg 3 was NG-monomethylarginine, a result consistent with that previously observed by mass spectrometry on H4 isolated from human 293T cells (see Example 2). No dimethylarginine was detected by mass spectrometry on rH4 after methylation in vitro by GST-PRMT1 using our assay conditions.

To determine if PRMT1 is responsible for mediating H4 Arg 3 activity in human 293T cells ectopically expressed PRMT1 isolated from these cells was investigated to determine if it retained the same substrate specificity for H4 as seen with recombinant PRMT1. To that end, HA-tagged PRMT1 (HA-PRMT1 wt), HA-tagged PRMT1 mutant (HA-PRMT1 mut)which lacks the putative AdoMet binding site or parent vector (HA)were ectopically expressed in human 293T cells followed by nuclear isolation and DNase I extraction. Histone methyltransferase (HMT)assays with these extracts using chicken core histones and AdoMet or ³H-AdoMet revealed that nuclear extracts from HA-PRMT1 expressing cells incorporated more ³H-AdoMet on H4 than extracts from either HA-PRMT1 mutant or HA expressing cells. Furthermore, immunoblots probed with the α-H4 R3Me antibody show that this activity is specific for histone H4 Arg 3. Immunoprecipitations of the same nuclear extracts with a HA specific antibody directly demonstrate that the enhanced H4 Arg 3 methyl activity is a result of HA-PRMT1 expression. These results indicate that cellular-derived PRMT1 can directly and specifically methylate Arg 3 on H4. Furthermore, given that expressed HA-PRMT1 is extracted by DNase I digestion, these data also suggest that PRMT1 is chromatin associated. It is also noted that expression of HA-PRMT1 enhances nucleosomal H4 Arg 3 methylation activity that is detectable in 293T DNase I nuclear extracts.

Although recombinant or cellular PRMT1 can methylate H2A, this methylation is not detected by the α-H4 R3Me antibody, suggesting that the α-H4 R3Me antibody is selective for H4 Arg 3 methylation. Since H4 Arg 3 methylation activity was detected in 293T control DNase I extracts, the following experiment was conducted to determine if the activity was due to endogenous PRMT1. To directly test this idea, endogenous PRMT1 was immuno-depleted from DNase I nuclear extracts and the extracts were then assayed for any remaining H4 methylation activity. Immuno-depletion of PRMT1 resulted in nearly a complete abolishment of H4 Arg 3 methylation activity in these extracts, indicating that PRMT1 is the major, if not exclusive, H4 Arg 3 HMT from human 293T cells. Furthermore, immunoprecipitated PRMT1 from the above immuno-depleted assay retained essentially all of the H4 HMT activity.

Materials and Methods

Cell Culture and Preparation of Nuclear Extracts and Histones

HeLa and 293T cells were grown at 37° C. in D-MEM containing 10% FBS or 5% FBS, respectively.Nuclei were isolated by detergent lysis and low speed centrifugation [Chen et al, J. Biol Chem 275, 40810 (2000)] followed by DNase I extraction or acid extraction of histones as previously described [One 100 mm plate of 293T cells (about 1.5×10⁶) were transfected with 4 μg of empty pCDNA vector or pCDNA-PRMT1 using the Effectene transfection reagent (Qiagen). Forty-eight hours after transfection, nuclei were isolated and core histones were purified by acid extraction and TCA precipitation]. Tetrahymena thermophila (strains CU 427 or CU 428)was grown in enriched 1% proteose peptone and macronuclear histones isolated from vegetatively growing cells as described by [K. Luger, T. J. Rechsteiner, T. J. Richmond. Methods Enzymology 304, 3 (1999)]. Chicken histones and nucleosomes were kindly provided by C. Mizzen. Yeast histones were isolated from the wild-type strain MX4-22A.

Expression Plasmids and Transfection

Bacterial and mammalian expression plasmids used for GST or HA expression of PRMT1 have been described elsewhere. The mammalian HA-PRMT1 putative AdoMet-binding mutant (SGT to AAA;amino acids 79-89) was generated by site-directed PCR mutagenesis. This construct was confirmed by sequencing. Transient transfections using 293T cells were performed using 15 μg of plasmid DNA on 100 mm dishes.

Methyltransferase Activity Assays

For histone methyltransferase (HMT)assays involving GST-PRMT1 or 293T DNase I nuclear extracts, 2 μg of core histones or 0.5 μg of recombinant H4 was incubated with 1 μg of GST-purified PRMT1 or 25 μg of 293T nuclear extract in histone methyltransferase (HMT) buffer (final concentration being 50 mM Tris-pH 8.0,1 mM PMSF and 0.5 mM DTT)along with 0.55 μCi of S-Adenosyl-L-[methyl-₃ H] methionine (₃H-AdoMet;72 Ci/mmol;NEN Life Science Products)for 30 min at 30° C. in a total volume of 10 μl. 2 μl of the reaction was spotted on Whatman P-81 for liquid scintillation counting to monitor reactions while the remainder analyzed by SDS-PAGE followed by Coomassie staining and fluorography. Identical reactions as described above were performed in parallel using non-radiolabeled AdoMet (15 μM final)and were analyzed by Western blotting using the α-H4 R3Me antibody.

Development of an Antibody Selective for Methylation of Arg 3 of Histone H4

A synthetic peptide coding for sequence 1-9 of the human H4 amino-terminus (SGRGKGGKGC*; SEQ ID NO: 15) in which the first serine was N-acetylated and residue 3 was made with asymmetric NG ,NG-dimethylarginine (Bachem) that was conjugated by standard protocols to keyhole limpet hemocyanin via a C-terminal artificial cysteine (C*) prior to rabbit immunization. Enzyme-linked immunosorbent assays (ELISAs) were performed as previously described using various amounts of unmodified or Arg 3 methylated H4 1-9 peptide as indicated in FIG. 1C to characterize antibody specificity from rabbit serum.

Immunoprecipitation Studies

DNase I nuclear extract from transiently transfected 293T cells were adjusted to 150 mM NaCl prior to immunoprecipitation. For α-HA immunoprecipitations, 50 μl of 293T DNasc I nuclear extract from transiently transfected 293T cells was incubated with 2.5 μl of α-HA antibody (HA.11;Covance) and 5 μl of protein G sepharose (Amersham)followed by incubation for 2 hr at 4° C. Immunoprecipitates were washed twice in RIPA buffer (50 mM Tris-HCl pH 7.4,150 mM NaCl, 1 mM EDTA, 1% Triton-X,0.1% SDS,1% Deoxycholate) followed by two washes in HMT buffer. For endogenous PRMT1 immunoprecipiation studies, 30 μl of 293T DNase I extract was adjusted to 150 mM NaCl and incubated with 1 μl α-PRMT1 and 3 μl of protein G sepharose (Amersham)followed by a 2 hr incubation at 4° C. Endogenous immunoprecipitates were washed three times in HMT buffer. Immunoprecitations were assayed for HMT activity as described above.

Western Blotting

Western blot analyses were performed using reagents and procedures from Amersham Life sciences. Rabbit α-H4 NG,NG-dimethylarginine 3 and α-PRMT1 was used at a dilution of 1:5,000. Monoclonal α-HA antibody was used at a dilution of 1: 1000.

Protein Microsequencing

For labeling studies involving GST-PRMT1 for microsequencing,the above HMT reaction volume,using chicken core histones as substrate, was scaled up 10-fold. Histones from this reaction were precipitated with TCA and H4 purified by RP-HPLC using a C8 column. Prior to sequencing,the N-terminus of H4 was deblocked [27].H4 was sequenced in an Applied Biosystems Model 477A Protein Sequencer with an in-line 120A PTH-Analyzer (Applied Biosystems) using optimized cycles. After conversion, 50% of the sample was transferred to the RP-HPLC for PTH-amino acid identification and the other 50% was collected for determination of radioactivity by scintillation counting.

Mass Spectrometry Analyses

H4 isolated from 293T cells was purified by RP-HPLC as described above before diluting to 2 pmol/μl with 50 mM ammonium bicarbonate, pH 8.5. Chymotrypsin (Roche)was added to 0.025 μg/μl and digestion carried out overnight at room temperature.2 pmol aliquots of the digest were loaded on a 360×75 um analytical column with 7 cm C18 beads (YMC ODS-AQ,Waters)and a ˜5 um emitter tip [28]for nano-HPLC microelectrospray ionization mass spectrometric analysis using an LCQ ion trap mass spectrometer (Finnigan). The HPLC gradient was 0-60% B in 70 minutes,60-100% B in 15minutes. Solvents A and B were 0.1M acetic acid in water and 0.1M acetic acid in 70% acetonitrile respectively. Mass spectrometric analyses involved targeted MS/MS of the +2 ions of the N-terminal chymotryptic fragment of H4 (SGRGKGGKGL; SEQ ID NO: 15). All possible combinations of N-terminal acetylation,S1 phosphorylation,K5 and K8 acetylation, and R3 methylation (mono-and di-)were analyzed. Arg 3 methylation was observed only in conjunction with N-terminal acetylation targeted MS/MS of the +3 ion was performed for confirmation.

### EXAMPLE 3

H4 Arg3 Methylation Mediated Events

Recent demonstrations that methylation on Lys 9 of H3 inhibits Ser 10 phosphorylation (S. Rea, et al., Nature 406, 593 (2000)) prompted investigation as to whether Arg 3 methylation interferes with acetylation of lysine residues on H4 tails. To this end, recombinant H4 that was either mock methylated or PRMT1 methylated was used as substrates for acetylation by p300 in the presence of ³H-Acetyl-CoA and the amount of acetylation on the two respective pools of H4 was compared. More particularly, recombinant H4 was purified and used as substrates for PRMT1 methylation in the presence of excess amounts of non-labeled SAM. Complete methylation was verified by the lack of further incorporation of ³H-SAM. Acetylation was performed in 20 μl volume containing 50 mM Hepes (pH 8.0), 5 mM DTT, 5 mM PMSF, 10 mM sodium butyrate, 10% glycerol, 2 ul ³H-acetyl-CoA and 2 ul of p300. The reaction mixture was incubated for 1 hr at 37° C. and terminated by the addition of SDS sample buffer.

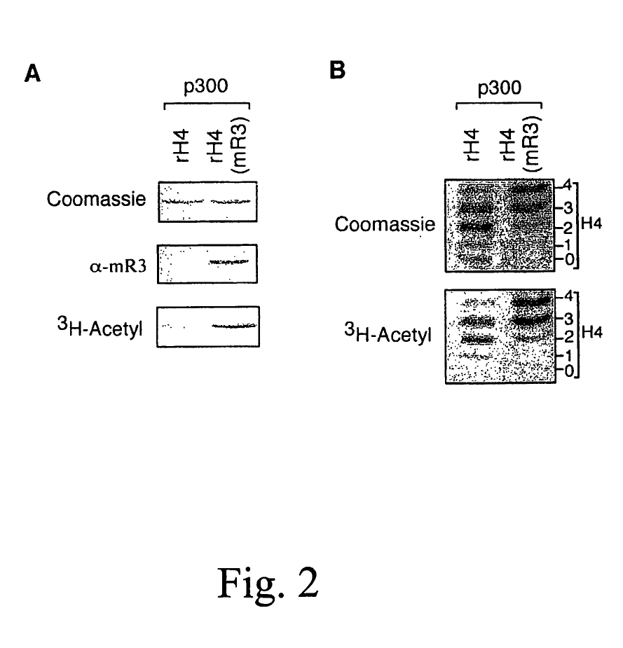

Methylation of H4 by PRMT1 stimulated its subsequent acetylation by p300 (FIG. 2A). To confirm this result, equivalent samples were analyzed with a Triton-Acetic Acid-Urea (TAU) gel, which separates different acetylated histone isoforms. The results demonstrate that PRMT1-methylated H4 is a better substrate for p300 when compared to non-methylated H4 because all H4 molecules were acetylated (no 0 acetylated form) by p300 (FIG. 2B). However, under the same conditions, a fraction of the mock methylated substrates still remain un-acetylated (0 acetylated form). To determine which of the four acetylable lysine residues are affected by Arg 3 methylation, the acetylation status of samples analyzed above was examined using acetylation site-specific antibodies. The results indicated that Arg 3 methylation facilitates K8 and K12 acetylation but has little affect on K5 or K16 acetylation.

To determine the effect of lysine acetylation on Arg 3 methylation, both hyperacetylated and hypoacetylated core histones were purified from HeLa cells and used as substrates for PRMT1 in the presence of ³H-SAM. After methylation, samples were resolved in a TAU gel followed by Coomassie staining and autoradiography. Only non- and mono-acetylated H4 isoforms were methylated to a detectable level although nearly equal amounts of the different H4 isoforms were present in the methylation reaction. Since non-acetylated H4 is the best substrate for PRMT1, when compared with different acetylated H4 isoforns, acetylation on lysine residues likely inhibits H4 methylation by PRMT1.

To determine whether this inhibition occurs in vivo, HeLa cells were treated with a histone deacetylase inhibitor, Tricostatin A (TSA), to induce hyperacetylation. Twelve hours after TSA treatment, core histones were isolated, and the methylation state of H4-Arg 3 was analyzed. Hypoacetylated H4 (untreated) had a higher Arg 3 methylation level when compared with hyperacetylated H4 (TSA treated) which had an almost undetectable Arg 3 methylation level. Therefore, hyperacetylation on lysine residues correlates with hypomethylation of H4 Arg 3. This result is consistent with the idea that acetylation on lysine residues inhibit subsequent Arg 3 methylation and it is also consistent with earlier studies demonstrating that H4 methylation preferentially occurs on non-acetylated histones while H3 methylation occurs preferentially on acetylated histones.

Since H4 contains four lysine residues that can be acetylated, we investigated whether acetylation on any of the four sites would have a similar affect on Arg 3 methylation. To this end, synthetic H4 tail peptides which were not acetylated, mono-acetylated, tri-acetylated and fully-acetylated, respectively, were used as substrates for PRMT1. Acetylation on any of the four lysines inhibited Arg 3 methylation by PRMT1. However, acetylation on Lys 5 had the most effect. In addition, acetylation on different lysines seemed to have an additive inhibition effect. Tri-acetylated and fully-acetylated peptides were severely impaired in serving as substrates for PRMT1.

Arg3 methylation enhanced lysine acetylation predicts that PRMT1 is likely to be involved in transcriptional activation. Indeed, PRMT1 has been shown recently to function as a co-activator of nuclear hormone receptors. However, its co-activator activity has not been linked to its HMT activity. To directly address the function of Arg 3 methylation on transcription, a single amino acid mutation (G80R) was introduced in the conserved SAM binding domain of PRMT1 which has been previously shown to impair its enzymatic activity (A. E. McBride et al, J Biol Chem 275, 3128 (2000). The ability of the mutant and wild-type PRMT1 to facilitate activation by androgen receptor (AR), which is known to use CBP/p300 as co-activators, was compared in chromatin context using Xenopus oocytes as a model system. A MMTV LTR based reporter was injected into the nuclei of Xenopus oocytes and successful assembly of the reporter into chromatin was confirmed by micrococcal nuclease digestion. Ectopic expression of AR in Xenopus oocytes led to an agonist-stimulated activation of the reporter. Co-expression of PRMT1 further augmented the activation by AR. Significantly, the PRMT1(G80R) mutant has little co-activator activity when compared with wild-type PRMT1. Western blot analysis revealed that the differences in transcription were not due to differential expression of PRMT1 and PRMT1(G80R) or their effect on AR expression. Therefore the HMT activity of PRMT1 is critical for its co-activator activity.

These studies demonstrate the interplay between Arg3 methylation and lysine acetylation and support the “histone code” hypothesis. The histone code hypothesis is based on the premise that histone proteins, and their associated covalent modifications, contribute to a mechanism that can alter chromatin structure, thereby leading to inherited differences in transcriptional “on-off” states or to the stable propagation of chromosomes by defining a specialized higher-order structure. H4 Arg 3 methylation is believed to play an important role in transcriptional activation. Interestingly, an H3-specific arginine methyltransferase CARM1 was also shown to function as a nuclear hormone receptor co-activator. Whether Arg 3 methylation helps the recruitment of specific HATs, such as p300, remains to be determined.

1. An antigenic peptide comprising an amino acid sequence of 5 to 20 amino acids wherein the amino acid sequence is selected from the group consisting of:

| | |
|---|---|
| Ser Gly Arg(M) Gly Lys, | (SEQ ID NO: 2) |
| Ser(P) Gly Arg Gly Lys, | (SEQ ID NO: 3) |
| Ser(P) Gly Arg(M) Gly Lys, | (SEQ ID NO: 4) |
| Ser Gly Arg Gly Lys(A), | (SEQ ID NO: 5) |
| Ser Gly Arg(M) Gly Lys(A), | (SEQ ID NO: 6) |
| and | |
| Ser(P) Gly Arg(M) Gly Lys(A). | (SEQ ID NO: 7) |

2. The antigenic peptide of claim 1 wherein the antigenic peptide consists of an amino acid sequence selected from the group consisting of

| | |
|---|---|
| Ser Gly Arg(M) Gly Lys Gly Gly Lys Gly, | (SEQ ID NO: 8) |
| Ser(P) Gly Arg Gly Lys Gly Gly Lys Gly, | (SEQ ID NO: 9) |
| Ser(P) Gly Arg(M) Gly Lys Gly Gly Lys Gly, | (SEQ ID NO: 10) |
| Ser Gly Arg Gly Lys(A) Gly Gly Lys Gly, | (SEQ ID NO: 11) |
| Ser Gly Arg(M) Gly Lys(A) Gly Gly Lys Gly, | (SEQ ID NO: 12) |
| Ser(P) Gly Arg(M) Gly Lys(A) Gly Gly Lys Gly, | (SEQ ID NO: 13) |
| and | |
| Ser Gly Arg Gly Lys Gly Gly Lys Gly. | (SEQ ID NO: 14) |

3. The antigenic peptide of claim 1 wherein the antigenic peptide consists of an amino acid sequence selected from the group consisting of

| | |
|---|---|
| Ser Gly Arg(M) Gly Lys Gly Gly Lys | (SEQ ID NO: 8) |
| Gly, | |
| Ser(P) Gly Arg(M) Gly Lys Gly Gly | (SEQ ID NO: 10) |
| Lys Gly, | |
| and | |
| Ser Gly Arg(M) Gly Lys(A) Gly Gly | (SEQ ID NO: 12) |
| Lys Gly. | |

4. A composition comprising the peptide of claim 1 and a pharmaceutically acceptable carrier.

5. The composition of claim 4 further comprising an adjuvant.

6. A purified antibody that specifically binds to a polypeptide comprising an amino acid sequence selected from the group consisting of:

| | |
|---|---|
| Ser Gly Arg Gly Lys, | (SEQ ID NO: 1) |
| Ser Gly Arg(M) Gly Lys, | (SEQ ID NO: 2) |
| Ser(P) Gly Arg Gly Lys, | (SEQ ID NO: 3) |
| Ser(P) Gly Arg(M) Gly Lys, | (SEQ ID NO: 4) |
| Ser Gly Arg Gly Lys(A), | (SEQ ID NO: 5) |
| Ser Gly Arg(M) Gly Lys(A), | (SEQ ID NO: 6) |
| and | |
| Ser(P) Gly Arg(M) Gly Lys(A). | (SEQ ID NO: 7) |

7. The antibody of claim 6 wherein the antibody specifically binds to a polypeptide comprising an amino acid sequence selected from the group consisting of

| | |
|---|---|
| Ser Gly Arg(M) Gly Lys Gly Gly Lys Gly, | (SEQ ID NO: 8) |
| Ser(P) Gly Arg Gly Lys Gly Gly Lys Gly, | (SEQ ID NO: 9) |
| Ser(P) Gly Arg(M) Gly Lys Gly Gly Lys Gly, | (SEQ ID NO: 10) |
| Ser Gly Arg Gly Lys(A) Gly Gly Lys Gly, | (SEQ ID NO: 11) |
| Ser Gly Arg(M) Gly Lys(A) Gly Gly Lys Gly, | (SEQ ID NO: 12) |
| Ser(P) Gly Arg(M) Gly Lys(A) Gly Gly Lys Gly | (SEQ ID NO: 13) |
| and | |
| Ser Gly Arg Gly Lys Gly Gly Lys Gly. | (SEQ ID NO: 14) |

8. The antibody of claim 7 wherein the antibody specifically binds to a polypeptide selected from the group consisting of

| | | |
|---|---|---|
| | Ser Gly Arg(M) Gly Lys Gly Gly Lys Gly, | (SEQ ID NO: 8) |
| | Ser(P) Gly Arg(M) Gly Lys Gly Gly Lys Gly, | (SEQ ID NO: 10) |
| and | Ser Gly Arg(M) Gly Lys(A) Gly Gly Lys Gly. | (SEQ ID NO: 12) |

9. The antibody of claim 8 wherein the antibody is a monoclonal antibody.

10. The antibody of claim 9, wherein the antibody specifically binds to the sequence Ser Gly Arg(M) Gly Lys Gly Gly Lys Gly Leu (SEQ ID NO: 8).

11. A composition comprising the antibody of claim 6 and a diluent or pharmaceutically acceptable carrier.

12. The antibody of claim 6 wherein said antibody is coupled to a bioactive substance selected from the group consisting of a drug, a toxin, a immunomodulator, a peptide effector and an isotope.

13. A diagnostic test kit for detecting euchromatin and heterochromatin, said kit comprising an antibody that specifically binds to a peptide selected from the group consisting of

| | | |
|---|---|---|
| | Ser Gly Arg(M) Gly Lys Gly Gly Lys Gly, | (SEQ ID NO: 8) |
| | Ser(P) Gly Arg(M) Gly Lys Gly Gly Lys Gly, | (SEQ ID NO: 10) |
| and | Ser Gly Arg(M) Gly Lys(A) Gly Gly Lys Gly. | (SEQ ID NO: 12) |

14. The kit of claim 13 comprising a first antibody that specifically binds to Ser(P) Gly Arg(M) Gly Lys Gly Gly Lys Gly (SEQ ID NO: 10), and a second antibody that specifically binds to Ser Gly Arg(M) Gly Lys(A) Gly Gly Lys Gly (SEQ ID NO: 12).

15. A method of detecting transcriptionally active regions of chromatin, said method comprising the steps of:
contacting a chromatin containing sample with an antibody that specifically binds to the sequence Ser Gly Arg(M) Gly Lys(A) Gly Gly Lys Gly (SEQ ID NO: 12);
removing unbound and non-specific bond antibody from the sample; and
detecting transcriptionally active regions of chromatin by identifying those regions of chromatin that have antibody bound to them.

16. The method of claim 15, wherein the antibody is labeled.

17. The method of claim 16, wherein the antibody is labeled with a fluorescent marker.

18. The method of claim 15, wherein the detection step comprises contacting said antibody with a labeled secondary antibody wherein said secondary antibody is an anti-immunoglobulin antibody.

19. A method of detecting mitotically active cells, said method comprising the steps of:
contacting a population of cells with an antibody that specifically binds to the sequence Ser(P) Gly Arg(M) Gly Lys Gly Gly Lys Gly (SEQ ID NO: 10);
removing unbound and non-specific bond antibody from the population of cells; and
detecting mitotically active cells by identifying those cells with antibody bound to them.

20. The method of claim 19, wherein the antibody is labeled.

21. The method of claim 20, wherein the antibody is labeled with a fluorescent marker.

22. The method of claim 19, wherein the detection step comprises contacting said antibody with a labeled secondary antibody wherein said secondary antibody is an anti-immunoglobulin antibody.

23. A method for detecting methylase activity in a sample said method comprising the steps of:
providing a sample that potentially contains methylase activity;
contacting the sample with a polypeptide comprising the sequence Arg Ser Gly Arg Gly Lys Gly Gly Lys Gly (SEQ ID NO: 2) for a predetermined length of time;
contacting the sample with the antibody of claim 6 after said predetermined length of time;
quantifying said antibody bound to said sample as an indication of the level activity of said methylase.

24. The method of claim 18 wherein said methylase is PRMT1.

25. A method of detecting chromatin alterations that are associated with a disease state, said method comprising the steps of
isolating chromatin from both normal and diseased tissue to create a first and second pool of chromatin;
contacting the first and second pools of chromatin with an antibody that specifically binds to a polypeptide selected from the group consisting of Ser(P) Gly Arg(M) Gly Lys Gly Gly Lys Gly (SEQ ID NO: 10) and Ser Gly Arg(M) Gly Lys(A) Gly Gly Lys Gly (SEQ ID NO: 12); and
comparing the staining pattern of the antibody bound chromatin isolated from normal tissue to the staining pattern of the antibody bound chromatin isolated from the diseased tissue.

26. The method of claim 25 further comprising the step of fragmenting the isolated chromatin before the immunoprecipitation step.

27. The method of claim 25 wherein the step of comparing the DNA comprises
immobilizing the DNA isolated from the first pool of chromatin onto a first solid surface;
immobilizing the DNA recovered from the second pool of chromatin onto a second solid surface;
probing the first and second solid surfaces with identical labeled nucleic acid sequences; and
identifying those sequences that bind only to the immobilized DNA isolated form the first pool of chromatin.


SEQUENCE LISTING (15 sequences)

SEQ ID NO: 1 - protein, 5 aa, Homo sapiens
SGRGK

SEQ ID NO: 2 - protein, 5 aa, Homo sapiens
  MOD_RES (3)..(3): METHYLATION
SGRGK

SEQ ID NO: 3 - protein, 5 aa, Homo sapiens
  MOD_RES (1)..(1): PHOSPHORYLATION
SGRGK

SEQ ID NO: 4 - protein, 5 aa, Homo sapiens
  MOD_RES (1)..(1): PHOSPHORYLATION
SGRGK

SEQ ID NO: 5 - protein, 5 aa, Homo sapiens
  MOD_RES (5)..(5): ACETYLATION
SGRGK

SEQ ID NO: 6 - protein, 5 aa, Homo sapiens
  MOD_RES (3)..(3): METHYLATION
SGRGK

SEQ ID NO: 7 - protein, 5 aa, Homo sapiens
  MOD_RES (1)..(1): PHOSPHORYLATION
SGRGK

SEQ ID NO: 8 - protein, 9 aa, Homo sapiens
  MOD_RES (3)..(3): METHYLATION
SGRGKGGKG

SEQ ID NO: 9 - protein, 9 aa, Homo sapiens
  MOD_RES (1)..(1): PHOSPHORYLATION
SGRGKGGKG

SEQ ID NO: 10 - protein, 9 aa, Homo sapiens
  MOD_RES (1)..(1): PHOSPHORYLATION
SGRGKGGKG

SEQ ID NO: 11 - protein, 9 aa, Homo sapiens
  MOD_RES (5)..(5): ACETYLATION
SGRGKGGKG

SEQ ID NO: 12 - protein, 9 aa, Homo sapiens
  MOD_RES (5)..(5): ACETYLATION
SGRGKGGKG

SEQ ID NO: 13 - protein, 9 aa, Homo sapiens
  MOD_RES (1)..(1): PHOSPHORYLATION
SGRGKGGKG

SEQ ID NO: 14 - protein, 9 aa, Homo sapiens
SGRGKGGKG

SEQ ID NO: 15 - protein, 10 aa, Homo sapiens
  MOD_RES (1)..(1): ACETYLATION
SGRGKGGKGC